(19) United States
(12) Patent Application Publication
(10) Pub. No.: US 2016/0166322 A1
(43) Pub. Date: Jun. 16, 2016

(54) Incise drape for surgical applications and method of positioning the drape over a patient

(71) Applicant: Matthew D. Lowrance, Lenoir City, TN (US)

(72) Inventor: Matthew D. Lowrance, Lenoir City, TN (US)

(21) Appl. No.: 14/544,228

(22) Filed: Dec. 10, 2014

(51) Int. Cl. A61B 19/08

(52) U.S. Cl. CPC A61B 19/08

(57) ABSTRACT

An incise drape positionable across an area of a patient upon which a surgical procedure is to be performed and an associated method of positioning the drape across the patient utilizes a thin flexible transparent sheet of plastic material having two side faces, and the thin flexible transparent sheet is provided with a pre-formed incision which extends between the two side faces of the sheet and which has slit portions which facilitate the formation of an opening in the sheet. A strip of removable material is adhesively secured to one side face of the sheet for maintaining the opposing edges of the incision together until the drape is placed across the patient for use. In addition, sighting indicia is printed upon the strip of material to facilitate the visual alignment of the center of the pre-formed incision with the area of the patient upon which the surgical procedure is to be performed.

### BACKGROUND OF THE INVENTION

This invention relates generally to drapes used in surgical applications relates, more particularly, to means and methods for draping an area of a patient prior to the undertaking of a surgical procedure desired to be performed upon the patient.

In preparation for some types of surgical procedures, such as may involve a relatively small target area of a patient, a transparent drape is placed over the target area of the patient, and an incision is formed, or cut, by an individual along the transparent drape. The opposing edges of the formed incision are then spread, or otherwise moved, apart to provide an opening in the drape through which the desired surgical procedure is to be performed. Heretofore, any incisions formed within such a transparent drape have been typically made by a surgeon who subsequently performs the surgical procedure.

It would be desirable to provide a new and improved incise drape which obviates the need that an incision be made in the drape after it has been placed over the patient.

Accordingly, it is an object of the present invention to provide a new and improved transparent drape of the aforedescribed class which has been provided with a pre-formed incision through which a surgical procedure can be performed and an associated method for positioning the drape over a target area of the patient.

Another object of the present invention is to provide such a drape whose pre-formed incision is shaped to facilitate the formation of an opening therein upon movement of opposing edges of the pre-formed incision away from one another to an out-of-the-way condition.

Still another object of the present invention is to provide such a drape whose pre-formed incision is bordered by opposing edges which are releasably held together until the opposing edges are desired to be spread, or moved, apart to expose an opening in the drape through which the surgical procedure can be performed.

Yet still another object of the present invention is to provide such a drape which can be readily secured across an opening provided in a sterile, or supplemental, drape which, in turn, is positionable so as to overlie the patient.

A further object of the present invention is to provide such a drape which is provided with a sighting guide which facilitates the alignment of the pre-formed incision of the drape in operative registry with a target, or desired, area of the patient as the drape is positioned across the patient.

A still further object of the present invention is to provide such a drape and an associated method which is particularly well-suited for use in cataract, refractive, retina, glaucoma or any other surgical procedure requiring a sterile ocular field.

One more object of the present invention is to provide such a method for positioning the drape of this invention across a patient which method, when used to position the drape across one of the patient's eyes, advantageously involves the use of the right hand when positioning the drape across the right eye of the patient and which advantageously involves the use of the left hand when positioning the drape across the left eye of the patient.

Still one more object of the present invention is to provide such a drape which is uncomplicated in structure, yet effective in operation.

### SUMMARY OF THE INVENTION

This invention resides in an incise drape positionable across an area of a patient upon which a surgical procedure is desired to be performed and an associated method for positioning the drape of this invention over one of the patient's eyes.

The drape of this invention includes a flexible transparent sheet defining a pre-formed incision which extends between the two side faces of the sheet and wherein the defined incision includes a primary slit portion which extends across one side face of the sheet and which has two opposite ends and two opposing edges which extend between the opposite ends. In addition, the incision further includes a first pair of secondary slit portions which are joined to one end of the primary slit portion so as to extend away from the opposing edges thereof and a second pair of secondary slit portions which are joined to the other end of the primary slit portion so as to extend away from the opposing edges thereof to facilitate the movement of the two opposing edges of the primary slit portion of the incision apart to form an opening in the sheet through which the desired surgical procedure can be performed.

The method of the invention includes the steps involved in positioning the incise drape of this invention over one eye of a patient upon which a surgical procedure is desired to be performed and wherein the sheet of the incise drape is provided with sighting indicia which facilitates the visual aligning of the defined incision in registry with the target area of the patient. The method includes the steps of folding the transparent sheet of the incise drape into a folded condition along a fold line which extends substantially along the length of the primary slit portion of the incision, and then holding the incise drape in the folded condition so that the sighting indicia is disposed adjacent said fold line of the transparent sheet. The incise drape is then placed, while in its folded condition, adjacent the face of the patient, and then the sighting indicia is visually aligned in operative registry with the one eye of the patient upon which the surgical procedure is desired to be performed. The sheet of the incise drape is then unfolded about its fold line, while the sighting indicia is maintained in operative registry with the one eye of the patient so that the sheet substantially returns to undeformed, planar condition across the patient's face and so that by subsequently moving the opposing edges of the primary slit portion apart to form an opening in the sheet through which the desired surgical procedure is to be performed, the formed opening is substantially aligned with the one eye of the patient.

### BRIEF DESCRIPTION OF THE DRAWINGS

FIG. 1 is a perspective view of an embodiment of an incise drape embodying features of the present invention and shown being utilized in an environment of intended use.

FIG. 2 is a perspective view similar to that of FIG. 1, but showing the incise drape being elevated above an underlying supplemental drape which is positionable across a patient.

FIG. 3 is a perspective view of the incise drape embodiment illustrated in FIG. 1.

FIG. 4 is a top plan view of the FIG. 3 embodiment with its strip of removable material being removed from the remainder of the embodiment.

FIG. 5 is a side elevation view of the FIG. 3 embodiment as seen from the right in FIG. 3.

FIG. 6 is a bottom plan view of the FIG. 3 embodiment showing a portion of one of its protective release strips being peeled from the remainder of the embodiment.

FIGS. 7-11 are perspective views illustrating, in sequence, steps involved in placing the FIG. 3 embodiment over a target, or desired, area of a patient and the formation of an opening in the embodiment.

FIG. 12 is a top plan view, similar to that of FIG. 4, of an alternative embodiment of an incise drape within which features of the present invention are embodied, shown without a strip of removable material covering the incision of the drape.

### DETAILED DESCRIPTION OF AN ILLUSTRATIVE EMBODIMENT

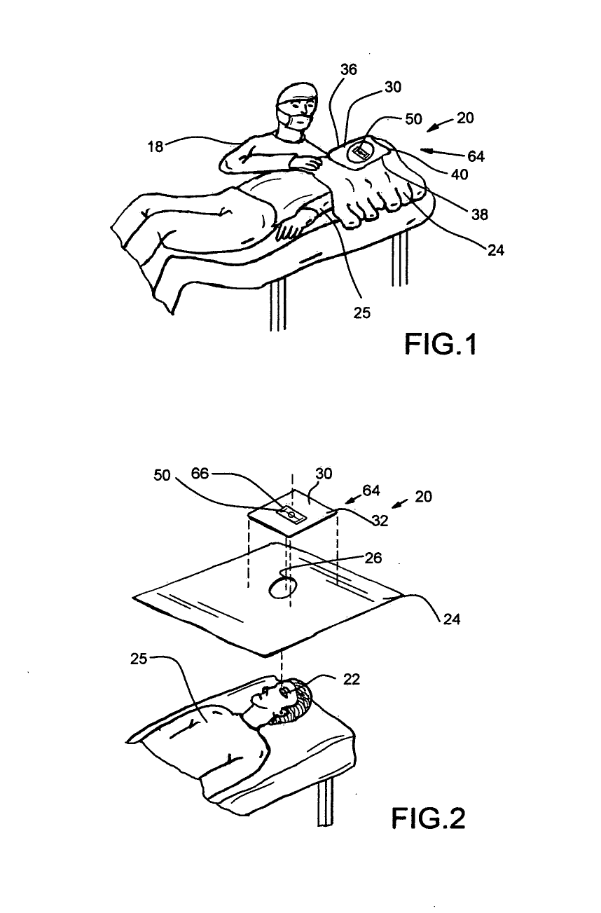

Turning now to the drawings in greater detail and considering first FIG. 1, there is illustrated an embodiment, generally indicated 20, of an incise drape within which features of the present invention are embodied shown being utilized in an exemplary environment of intended use. More specifically, the depicted FIG. 1 environment includes a patient 25 upon which a surgical procedure is desired to be performed by a surgeon 18 and a sterile (e.g. cloth), or supplemental, drape 24 which is positionable across the patient 25 with the embodiment 20. Furthermore and inasmuch as it is the eye of the patient 25 which, in the depicted example, is to be operated upon, the patient 25 lies upon his back with his face directly upwardly. As best shown in FIG. 2, the supplemental drape 24 is sized to cover a relatively large area of the patient 25 and is provided with a preformed opening 26 across which the incise drape 20 is secured. Although the opening 26 of the supplemental drape 24 can take any of a number of shapes, the opening 26 of the depicted supplemental drape 24 is substantially oval in shape.

In preparation for the desired surgical procedure and with reference to FIGS. 1 and 2, the incise drape 20 is positioned upon and secured to the supplemental drape 24 so as to span the drape opening 26, and then the supplemental drape 24 is positioned across the patient 25 so that its opening 26 (and the incise drape 20 secured thereacross) is positioned substantially centrally over a target area of the patient 25 upon which, or through which, a surgical procedure is to be performed.

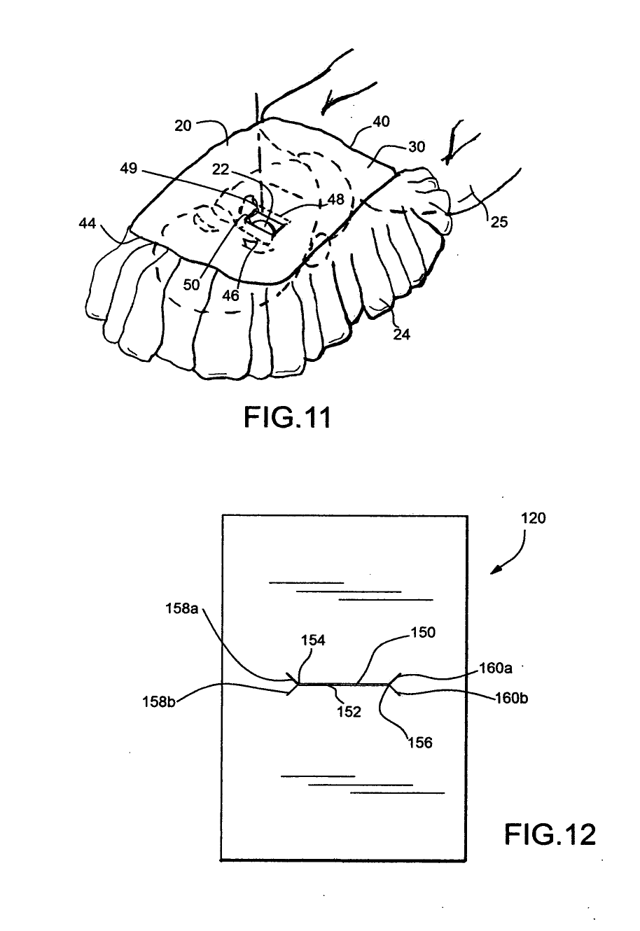

As will be described in greater detail herein, the incise drape 20 is provided with a preformed incision 50 which is shaped so that opposing edges (described herein) of the incision 50 can be readily moved away from one another to provide a sizeable opening 49 (FIG. 11) in the drape 20 through which the surgical procedure is carried out. Additional features of the drape 20 maintain the opposing edges of the incision 50 together until the opening 49 is desired to be formed in the drape 20 and help a user (e.g. the FIG. 1 surgeon 18) to accurately position the drape 20 over the patient 25.

Furthermore, the oval-shaped opening 26 of the supplemental drape 24 is substantially centered over the desired, or target, area of the patient 25 at which the surgical procedure is desired to be performed, and the incise drape 20 is, in turn, positioned over the oval-shaped opening 26 and secured to the underlying drape 24 (in a manner described herein) at locations adjacent the boundary, or edges, of the oval-shaped opening 26.

Within the depicted FIG. 1 environment, the surgeon 18 is preparing for surgery to be performed upon one eye (i.e. the right eye), indicated 22 in FIG. 2, of the patient 25. Therefore and within the FIG. 1 environment, it is the one eye which provides the target area 22 over which the incision 50 of the incise drape 20 is substantially centered for the subsequent surgical procedure. As will be apparent herein, the incise drape 20 is adaptable and well-suited for use in cataract, refractive, retina, glaucoma and any other surgical procedure which requires a sterile ocular field. It will be understood, however, that although the incise drape 20 is described and illustrated herein as being usable in conjunction with surgical procedures which relate to a patient's eye, the incise drape 20 can find use in other classes of surgical procedures involving a relatively small target area of a patient. Accordingly, the principles of the present invention can be variously applied.

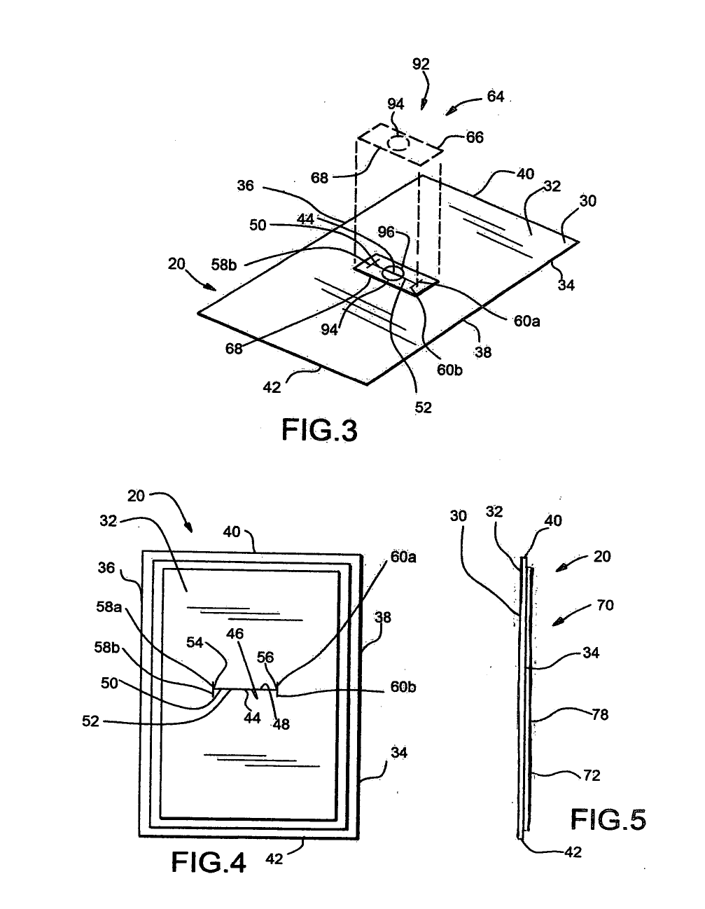

With reference to FIGS. 3-6, the incise drape 20 includes a relatively thin, flexible, transparent plastic sheet 30 of substantially rectangular shape having two side faces 32, 34 wherein one side face 32 is referred to herein as the top face of the sheet 30 and the other side face 34 is referred to herein as the bottom face of the sheet 30. Defined along the outer boundary of the transparent sheet 30 are a first pair of linear edges 36 and 38 which extend along two of the opposite sides of the sheet 30 and a second pair of linear edges 40 and 42 which extend along the remaining two of the opposite sides (i.e. the top and bottom) of the sheet 30.

When the incise drape 20 is used within the FIG. 1 environment, the drape 20 is oriented over the patient 25 so that its linear edges 36, 38 are oriented lengthways along the length of the patient 25. Accordingly, the linear edges 36, 38 of the sheet 30 are preferably longer than are the linear edges 40, 42 thereof. By way of example, the linear edges 36, 38 of the sheet 30 of the depicted drape 20 can be about fourteen cm in length, and the linear edges 40, 42 of the sheet 30 can be about ten cm in length.

The material out of which the sheet 30 of the depicted drape 20 is constructed is comprised of a polyvinyl acetate/ethyl vinyl acetate blend, but other materials can be used as long as the resultant sheet 30 is transparent and flexible and possesses a degree of elasticity.

It is a feature of the drape 20 that its sheet 30 includes a pre-formed incision 50, introduced earlier, which extends between the top and bottom faces 32, 34 of the sheet 30 so that the geometric center, indicated 44, of the incision 50 is disposed substantially centrally of the top and bottom faces 32, 34 of the sheet 30. With the geometric center 44 of the incision 50 disposed substantially centrally of the sheet 30, the incision 50 is disposed substantially centrally of the sheet 30, as well. Meanwhile, it is a feature of the incision 50 that it includes a primary slit portion 52 which extends across a major section of the sheet 30 and terminates at opposite ends, indicated 54 and 56, of the primary slit portion 52. Furthermore, there is provided on opposite sides of the slit portion 52 two opposing edges 46, 48 which extend between the opposite ends 54 and 56 of the primary slit portion 52.

With reference still to FIGS. 3 and 4, the incision 50 also includes a first pair 61 of a secondary slit portions 58a, 58b which are joined to the primary slit portion 52 at one end 54 thereof wherein the secondary slit portions 58a, 58b extend away from the opposing edges 46 and 48 on opposite sides thereof. Moreover, the incision 50 also includes a second pair 63 of secondary slit portions 60a, 60b which are joined to the primary slit portion 52 at the other end 56 thereof and wherein the secondary slit portions 60a, 60b extend away from the opposing edges 46 and 48 on opposite sides thereof.

The incision 50 of the depicted incise drape 20 is substantially I-shaped in form in that its primary slit portion 52 extends along a substantially linear path across the side faces 32 and 34 of the sheet 30, and each secondary slit portion 58a, 58b, 60a, 60b of the first and second pairs 61 and 63 of secondary slit portions extends along a substantially linear path from the end 54 or 56 of the primary slit portion 52 to which the secondary slit portion 58a, 58b, 60a, 60b is joined and is oriented at a substantially right angle to the path of the linear slit portion 52 so as to extend transversely across the side faces 32, 34 of the sheet 30. Furthermore and as best shown in FIG. 4, the secondary slit portions 58a, 58b, 60a, 60b are substantially of equal length.

The primary slit portion 52 is bordered on each of its two sides by two opposing edges, indicated 46, 48 in FIG. 4. As will be apparent herein, when the two opposing edges 46, 48 are spread apart (e.g. manually), or otherwise moved away from one another, to form the FIG. 11 opening 49, the secondary slit portions 58a, 58b, 60a, 60b permit the edges 46, 48 to be spread further apart than would be the case if the secondary slit portions were not formed within the sheet 30. In other words, the secondary slit portions 58a, 58b, 60a, 60b permit the opposing edges 46, 48 to be spread apart by an appreciable extent with no resistance from the sheet 30 at the ends 54, 56 of the primary slit portion 52. This being the case, neither the primary slit portion 52 nor the secondary slit portions 58a, 58b, 60a, 60b need to be formed within the sheet 30 by a user (e.g. the FIG. 1 surgeon 18) once the drape 20 of has been positioned over the patient 25, and the pre-formed incision 50 is advantageous in this respect. Moreover, the spreading apart of the opposing edges 44 and 46 of the incision 50 creates a pair of flaps which can be, if desired, readily folded beneath the remainder of the sheet 30 to create a barrier to the lashes and conjunctiva which is where the majority of the pathogens that can cause endophthalmitis are located.

If the size of the opening 49 were desired to be enlarged to, for example, conform to the size of larger orbits or the variance in the shape or depth of the orbit and/or the nasal bridge of the patient 25, the user will likely spread the opposing edges 46, 48 of the incision 50 further apart than the extent allowed by the secondary (e.g. transversely-extending) slit portions 58a, 58b, 60a, 60b. If such were to occur, tears will form in the sheet 30 wherein such formed tears lengthen the secondary slit portions 58a, 58b, 60a, 60b to thereby enlarge the opening 49. In other words, the existence of the secondary slit portions 58a, 58a, 60a, 60b ensures that any tearing of the sheet 30 to forcibly enlarge the opening 49 is initiated at the free, or outwardmost, ends of the secondary slit portions 58a, 58b, 60a, 60b. Therefore, the secondary slit portions 58a, 58b, 60a, 60b provide paths along the sheet 30 at which any such tears are predictably formed (so as to increase the length of the secondary slit portions 58a, 58b, 60a, 60b) and are further advantageous in that they permit the opening 49 to be readily enlarged, as needed, without the need for scissors.

It is also a feature of the drape 20 that it includes means, generally indicated 64 In FIG. 3, for preventing the separation of the opposing edges 46, 48 of the incision 50 before the drape 20 is positioned in place over the patient 25. Within the depicted 20, the preventing means 64 is in the form of a strip of removable material 66 (e.g. a flexible plastic material) which bears an amount of adhesive 68 (e.g. a low-tack adhesive) along one side face thereof and is positioned adhesive-face down against the top face 32 of the sheet 30 so as to extend along a major section of (e.g. preferably, the entirety of) the length of the primary slit portion 52. With the strip of removable material 66 extending along the linear slit portion 52 in such a manner, the sections of the sheet 30 which extend along each of the opposing edges 46, 48 are engaged by the adhesive 68 so that the strip 66 holds the opposing edges 46, 48 of the incision 50 together in a stationary condition adjacent one another.

Once the drape 20 is positioned in place over the patient 25 in preparation of the surgical procedure desired to be performed, one end (e.g. either end) of the strip 66 can be grasped by an individual (e.g. the FIG. 1 surgeon 18) and lifted from the sheet 30 to thereby peel the strip 66 from the top face 32 thereof. Once the strip 66 is removed from the sheet 30, the opposing edges 46, 48 of the incision 50 are free to be manually pushed away from one another to form the opening 49. For a reason which will be apparent herein, the strip of removable material 66 is transparent.

In practice, the strip of release material 66 serves to rigidify the region of the sheet 30 adjacent the incision 50. In other words and without the strip 66 being positioned along the length of the primary slit portion 52, the sheet 30 or, more specifically, the regions of the sheet 30 adjacent the opposing edges 46, 48 of the incision 50 might be too flimsy (due to the separation of the sheet 30 along the slit portions 52, 58a, 58b, 60a and 60b) to permit the drape 20 to be accurately positioned over the target area 22 without difficulty. However, the securement of the strip 66 along the primary slit portion 52 as aforedescribed reduces the flimsiness in the sheet 30 along the incision 50 and thereby allows for easier manipulation of the sheet 30 adjacent the target area 22. Moreover, the ease with which the strip 66 can be removed from the sheet 30 provides for relatively quick and easy access to the incision 50.

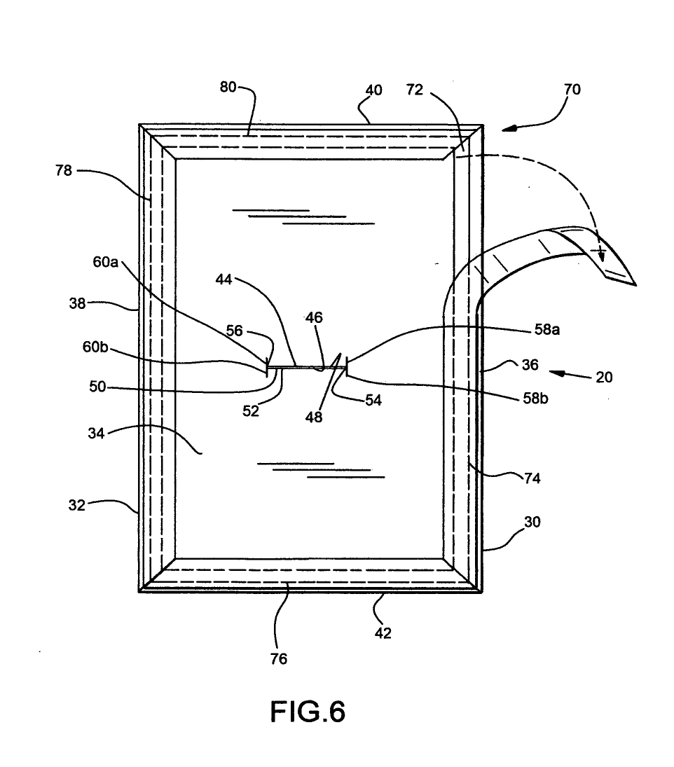

It is also a feature of the drape 20 that it includes means, generally indicated 70 in FIGS. 5 and 6, for securing the sheet 30 to the supplemental drape 24 upon placement of the bottom face 34 of the sheet 30 into contact with the supplemental drape 24. Within the depicted drape 20, the securing means 70 is in the form of an amount of adhesive 72 which is arranged in a plurality of strips 74, 76, 78 and 80 which extend along the four linear edges 36, 38, 40, 42 of the sheet 30 on the bottom face 34 thereof. When placing the incise drape 20 upon the supplemental drape 24, the incise drape 20 is directed bottom face-first downwardly upon the supplemental drape 24, and upon engagement of the underlying strips 74, 76, 78 and 80 of adhesive 72 with the upper surface of the supplemental drape 24, the incise drape 20 becomes adhesively secured in a stationary relationship with the supplemental drape 24 and the incise drape 20 is thereby held in place across the supplemental drape 24 throughout the surgical procedure performed upon the patient 25.

Preferably and as best shown in FIG. 6, each adhesive strip 74, 76, 78 and 80 is covered with protective strips 84, 86, 88 and 90 of release paper which protect the adhesive strips 74, 76, 78 and 80 until the drape 20 is desired to be positioned in a stationary condition atop the supplemental drape 24. It follows that prior to the placement of the incise drape 20 atop the supplemental drape 24, the release paper strips 84, 86, 88 and 90 are peeled, and thus removed, from the strips 74, 76, 78 and 80 of adhesive 72 to thereby expose the adhesive strips 74, 76, 78 and 80. Once the adhesive strips 84, 86, 88 and 90 are exposed, the bottom face 34 of the sheet 30 can be subsequently pressed into engagement with a surface (e.g. an upper surface) of the underlying supplemental drape 24 to adhesively secure the sheet 30 in a stationary condition atop the supplemental drape (and across the oval opening 26 thereof) so that the sheet 30 is thereafter prevented from slipping or dislodging from the supplemental drape 24 during a surgical procedure.

In practice and with reference again to FIG. 2, the incise drape 20 is first secured to the surface of supplemental drape 24 so as to cover the oval opening 26 provided therein, and then the supplemental drape 24, with the incise drape 20 adhesively secured thereto, is then placed in overlying relationship across the patient 25 so that the incise drape 20 is disposed over the target area 22 of the patient 25 at which the surgical procedure is to be performed. By securing the incise drape 20 to the supplemental drape 24 before the drape 24 is placed over the patient 25, the incise drape 20 can be pressed firmly against the supplemental drape 24 to secure the two together as a unitary structure without causing harm to the patient 25.

It is also a feature of the incise drape 20 that it includes a sighting guide, generally indicated 92 in FIG. 3, which facilitates the centering of the incise drape 20 over the target area 22 of the patient 25 upon which, or at which, the surgical procedure is desired to be performed. Although the sighting guide 92 can take any of a number of forms, the guide 92 of the depicted drape 20 includes indicia 94 in the form of a relatively small circular dot, indicated 96 in FIG. 3, which is printed upon the strip of removable material 66 and disposed so as to be centered over the midpoint, indicated 44 in FIG. 4, of the primary slit portion 52. Furthermore, the circular dot 96 is not so opaque that the user (e.g. the FIG. 1 surgeon 18) cannot see through the strip of removable material 66. Further still, the dot 96 is preferably sized (i.e. in diameter) to correspond to the corneal diameter of the patient's eye 22, but a dot 96 which possesses an alternative diameter can be had.

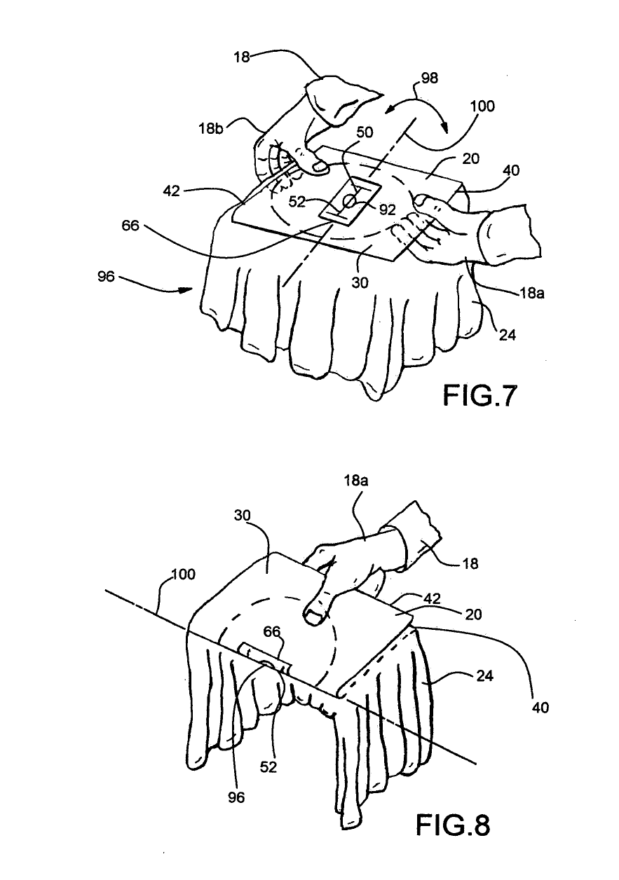

In a preferred method of positioning the drape 20 across the target area 22 of the patient 25 with the aid of the sighting guide 92 and with reference to FIG. 7, the drape 20 is gently folded (or rolled) over upon itself (by, for example, moving the opposite edges 40, 42 of the sheet 30 toward one another along the direction of the arrow 98 so that one edge 42 overlies the other edge 40, as best shown in FIG. 8) and so that the primary slit portion 52 of the incision 50 extends substantially along the line of fold, indicated 100 in FIGS. 7 and 8, about which the sheet 30 is folded. Such a disposition of the primary slit portion 52 along the fold line substantially centers the circular dot 96 of the sighting guide 92 along the length of the fold line 100 and so that when the drape 20 is viewed from one side of the folded arrangement, one half of the circular dot 96 is viewable, as best shown in FIG. 8.

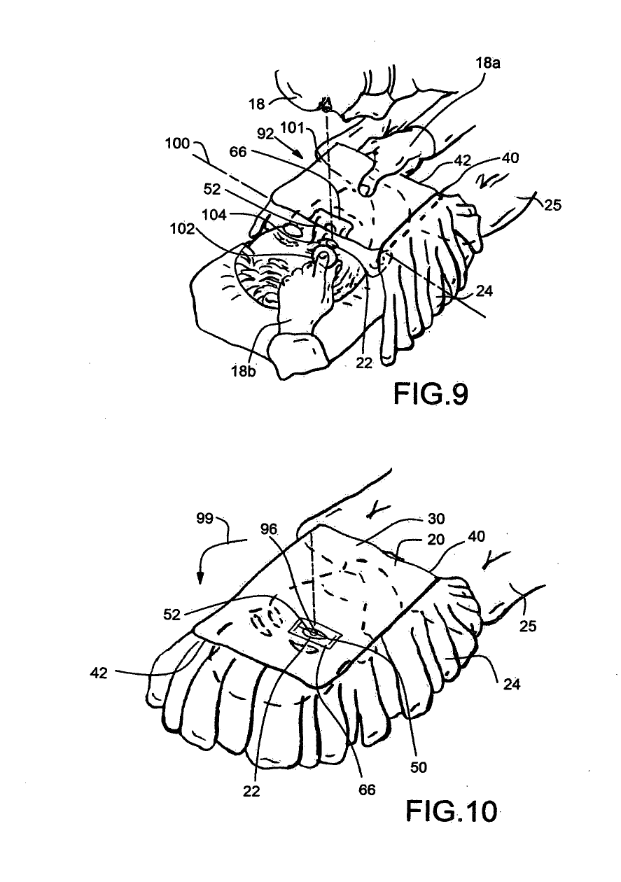

The sheet 30 is depicted in its folded condition in FIG. 8 while being held in such a folded condition by the user with only one hand, indicated 18a. With the drape 20 held in its FIG. 8 folded condition with the hand 18a, the drape 20, with the supplemental drape 24 secured thereto, is then positioned over the patient 25 so that the circular dot 96 (i.e. the one-half of the circular dot 96 which is viewable from one side thereof) is operatively aligned with the target area 22 as depicted in FIG. 9. In other words, the sheet 30, when in its folded condition, is manipulated with the hand 18a, as necessary, so that the center of the circular dot 96 (disposed along the fold line 100) is positioned in operative (e.g. aligned) registry with the center of the target area 22 of the patient 25 through which, or at which, the surgical operation is desired to be performed. This alignment (e.g. vertical alignment) of the center of the circular dot 96 with the target area 22 of the patient 25 is performed visually along, for example, the vertical line of sight, indicated 101 in FIG. 9.

Upon positioning the center of the dot 96 in operative registry with the center of the target area 22 at which the surgical procedure is desired to be performed, the sheet 30 is then unfolded (or unrolled) to its undeformed, planar condition, as shown in FIG. 10, by moving the sheet edge 42 relative to the sheet edge 40 along the direction of the FIG. 10 arrow 99 while the sheet edge 40 (i.e. depicted as the lower edge 40 in FIG. 9) is maintained in a stationary condition over the patient 25. Such an unfolding of the sheet 30 in this manner positions the geometric center, or midpoint (FIG. 4), of the incision 50 directly over the center of the target area 22. Thus, the opening 49 which is subsequently formed in the sheet 30 by spreading apart the opposing edges 46, 48 of the primary slit portion 52 will thereby be centered over the target area 22 through which, or at which, the surgical procedure is desired to be performed.

For purposes of enlarging the target area 22 to facilitate the aligning of the circular dot 96 with the target area 22 in the aforedescribed manner, it may be desirable to retract the upper and lower eyelids of the eye of the patient 25 upon which a surgical procedure is desired to be performed prior to the unfolding of the sheet 30 to its FIG. 10 undeformed, planar condition. If such is the case and with reference again to FIG. 9, while one hand 18a is used to grasp and hold the sheet 30 of the drape 20 in its FIG. 9 folded condition, the other hand, indicated 18b, of the user is free to grasp and hold a soft object, such as a cotton swab 102, to retract the upper eyelid, indicated 104, of the patient's eye. In other words, the upper eyelid 104 can be manipulated to a retracted condition with the cotton swab 102 held within the user's hand 18b. In the meantime, one side of the folded drape 20 (i.e. the side of the drape 20 disposed closest to the patient 25) and held with the user's hand 18a is used to manipulate the lower eyelid of the patient to a retracted condition. By enlarging the size of the target area 22 in such a manner, the circular dot 92 can be more easily and visually aligned (e.g. vertically aligned) with the center of the eye.

Once the sheet 30, or more specifically, the circular dot 96 of the strip 66 is positioned in aligned (e.g. vertical) registry with the center of the target area 22 of the patient 25 as depicted in FIG. 10, the strip 66 is manually removed, or peeled, from the sheet 30 to thereby release the opposing edges 46, 48 from the their secured condition adjacent one another. At that point, the opposing edges 46, 48 (FIG. 4) of the primary slit portion 52 can be pushed downwardly by the user (and thus away from one another) and thereby folded beneath the remainder of the sheet 30 so that the edges 46, 48 are thereby moved to out-of-the-way conditions, as illustrated in FIG. 11, to provide the opening 49 (of substantially rectangular shape) through which the desired surgical procedure is subsequently performed.

Upon formation of the opening 49, the globe of the eye 22 is exposed to the surgeon 18 and ready for speculum placement. It follows that the secondary transversely-extending slit portions 58a, 8b, 60a, 60b accommodate the spreading apart of the opposing edges 46, 48 of the linear slit portion 52 away from one another without the need that comparable (e.g. transversely-extending) slits be formed (e.g. cut with scissors) at the opposite ends 54, 56 of the linear slit portion 52 after the sheet 30 has been positioned in place over the patient 25.

The aforedescribed method of positioning the drape 20 over the target area 22 of the patient 25 is advantageous in that it is performed while holding the sheet 30 in a folded condition with one hand. More specifically and in order to align the circular dot 96 with the right eye of the patient 25, the sheet 30 is preferably moved upwardly along the face of the patient 25 (from inferior eye) while the sheet 30 is held in its folded condition with the user's right hand. Similarly and in order to align the circular dot 96 with the left eye of the patient 25, the sheet 30 is moved upwardly along the face of the patient 25 while the sheet 30 is held in its folded condition with the user's left hand.

Therefore and to help ensure that it is the correct eye (i.e. the right eye versus the left eye) of the patient 25 which is ultimately operated upon by the surgeon 18, the individual who positions the drape 20 into operative registry with the correct eye uses the corresponding hand to hold the hand 30 in its folded condition. That is to say, that if it is the right eye which is to be operated upon through the drape 20, the user takes care to place the sheet 30, when in its folded condition, into overlying relationship with the patient's face with the user's right hand. Conversely, if it is the left eye which is to be operated upon through the drape 20, the user takes care to place the sheet 30, when in its folded condition, into overlying relationship with the patient's face with the user's left hand. Accordingly, applicant's method helps to ensure that the correct eye (i.e. the right eye versus the left eye) is operated upon by the surgeon.

Exemplary dimensions of the sheet 30 of the drape 20 are provided here as follows: The length of the edges 40, 42 of the sheet 30 can range from about 10 cm to 15 cm; the length of the edges 36, 38 of the sheet 30 can range from about 14 cm to about 19 cm; the primary slit portion 52 can be about 41 mm long; and the length of the secondary slit portions 56 or 58 can be about 8 mm in length.

It will be understood that numerous modifications and substitutions can be had to the aforedescribed embodiment 20 of FIGS. 1-11 without departing from the spirit of the invention. For example, although the incision 50 of the depicted drape 20 of FIGS. 1-11 is I-shaped in form having secondary slit portions 56, 58 are shown and described as extending transversely across the sheet 30 at generally right angle with respect to the primary slit portion 52, the secondary slit portions need not be oriented at right angle with respect to the primary slit portion 52. For example and with reference to FIG. 12, there is shown an alternative embodiment, generally indicated 120, of a drape having an incision 150 having a primary slit portion 152 and a secondary slit portions 158a, 158b, 160a, 160b which collectively provide a V-shaped slit at each end 154, 156 of the primary slit portion 152. Therefore and within the incision 50 or 150 of either embodiment 20 of FIGS. 1-11 or the embodiment 120 of FIG. 12, the secondary slit portions extend away from the opposite sides of the primary slit portion 52 or 152 to which the secondary slit portions are joined.

Accordingly, the aforedescribed embodiment 20 is intended for the purpose of illustration and not as limitation.

1. An incise drape positionable across an area of a patient upon which a surgical procedure is desired to be performed, the drape comprising:
a thin flexible transparent sheet of plastic material having two opposite side faces
wherein the thin flexible transparent sheet defines a pre-formed incision which extends between the two side faces of the sheet and wherein the defined incision includes a primary slit portion which extends across one side face of the sheet and which has two opposite ends and two opposing edges which extend between the opposite ends; and
the incision further includes a first pair of secondary slit portions which are joined to one end of the primary slit portion so as to extend away from the opposing edges thereof and a second pair of secondary slit portions which are joined to the other end of the primary slit portion so as to extend away from the opposing edges thereof to facilitate the movement of the two opposing edges of the primary slit portion of the incision apart to form an opening in the sheet through which the desired surgical procedure can be performed.

2. The incise drape as defined in claim 1 wherein the incision is located substantially centrally of the two side faces of the sheet.

3. The incise drape as defined in claim 1 wherein the primary slit portion extends along a substantially linear path across the one side face of the sheet, and each secondary slit portion of the first and second pairs of the secondary slit portions extends along a substantially linear path from the end of the primary slit portion to which it is joined.

4. The incise drape as defined in claim 3 wherein the primary slit portion has a length which falls in the range of between about 20 and 60 mm, and each of the secondary slit portions is at least about 8 mm in length.

5. The incise drape as defined in claim 1 further comprising a strip of removable material which is adhesively secured to the one side face of the sheet so as to extend along a major section of the primary slit portion to maintain the two opposing edges thereof in a stationary condition with respect to one another until the incise drape is positioned across the area of the patient.

6. The incise drape as defined in claim 5 wherein the strip of removable material is sized to adhesively cover substantially the entirety of the defined incision.

7. The incise drape as defined in claim 5 wherein the primary slit portion includes a midpoint disposed substantially midway along the length of the primary slit portion and the strip of removable material includes a transparent peel strip portion and sighting indicia which is printed upon the transparent strip portion, and the peel strip portion is positioned along the defined incision so that the sighting indicia is substantially aligned with the midpoint of the primary slit portion.

8. The incise drape as defined in claim 7 wherein the sighting indicia includes a circular dot which is substantially centered with the midpoint of the primary slit portion.

9. The incise drape as defined in claim 7 wherein the peel strip portion is elongated in shape with two opposite ends, and at least one of the two opposite ends of the peel strip portion is capable of being gripped for removal of the peel strip portion from the sheet.

10. The incise drape as defined in claim 1 wherein the sheet is substantially rectangular in shape.

11. The incise drape as defined in claim 1 wherein the defined incision is substantially I-shaped in form.

12. The incise drape as defined in claim 1 further comprising an amount of adhesive which is borne on a side face of the sheet facilitating the securement of the sheet in a stationary condition across the patient during the performance of a surgical procedure.

13. The incise drape as defined in claim 12 wherein the patient upon which a surgical procedure is to be performed is desired to be overlain by a supplemental drape adapted to cover a larger area of the patient than is capable of being covered by the incise drape, and the adhesive borne by the incise drape enables the incise drape to be adhesively secured to the supplemental drape so that both the incise and supplemental drapes can be placed as a single unit across the area of the patient upon which a surgical procedure is desired to be performed.

14. The incise drape as defined in claim 12 wherein the sheet has outer edges, and the amount of adhesive borne by the one side face is arranged in strips of adhesive which extend along the outer edges of the sheet.

15. The incise drape as defined in claim 14 further comprising strips of removable material which cover, and thereby protect, the strips of adhesive until the strips of adhesive are desired to be exposed.

16. An incise drape positionable across a target area of a patient at which, or through which, a surgical procedure is desired to be performed, the drape comprising:
a thin flexible transparent sheet of plastic material having two opposite side faces wherein the thin flexible transparent sheet is provided with a pre-formed incision which extends between the two side faces of the sheet and includes a primary slit portion which extends across one side face of the sheet and which has two opposing edges which extend between the two opposite sides; and
the incision further includes a first pair of secondary slit portions which are joined to one end of the primary slit portion so as to extend away from the opposing edges thereof and a second pair of secondary slit portions which are joined to the other end of the primary slit portion so as to extend away from the opposing edges thereof to facilitate the movement of the two opposing edges of the primary slit portion of the incision apart to form an opening in the sheet through which the desired surgical procedure can be performed; and
sighting indicia associated with the sheet which facilitates the visual aligning of the defined incision in registry with the target area of the patient.

17. The incise drape as defined in claim 16 wherein the incision includes a midpoint which is located substantially centrally of the primary slit portion, and the incise drape further includes a strip of removable material including a transparent strip portion which is adhesively secured along a major section of the length of the primary slit portion, and the sighting indicia is printed upon the transparent strip portion, and the transparent strip portion is positioned along the primary slit portion so that the sighting indicia is substantially aligned with the midpoint of the primary slit portion.

18. The incise drape as defined in claim 17 wherein the sighting indicia includes a circular dot which is printed upon the transparent strip portion.

19. The incise drape as defined in claim 16 further comprising an amount of adhesive which is borne on a side face of the sheet facilitating the securement of the sheet in a relatively stationary condition across the patient during the performance of a surgical procedure.

20. A method for positioning an incise drape over one eye of a patient upon which a surgical procedure is desired to be performed, the method comprising the steps of:
providing an incise drape including a thin flexible transparent sheet of plastic material which is substantially planar when in an undeformed condition and having two opposite side faces wherein the thin flexible transparent sheet is provided with a pre-formed incision which extends between the two side faces of the sheet and includes a primary slit portion which extends across one side face of the sheet and which has two opposing edges which extend between the two opposite sides, and wherein the incision further includes a first pair of secondary slit portions which are joined to one end of the primary slit portion so as to extend away from the opposing edges thereof and a second pair of secondary slit portions which are joined to the other end of the primary slit portion so as to extend away from the opposing edges thereof to facilitate the movement of the two opposing edges of the primary slit portion of the incision apart to form an opening in the sheet through which the desired surgical procedure can be performed, and wherein the incise drape further includes sighting indicia associated with the sheet which facilitates the visual aligning of the defined incision in registry with the target area of the patient;
folding the transparent sheet of the incise drape into a folded condition along a fold line which extends substantially along the length of the primary slit portion of the incision;
holding the incise drape in the folded condition so that the sighting indicia is disposed adjacent said fold line of the transparent sheet;
placing the incise drape, while in its folded condition, adjacent the face of the patient;
visually aligning the sighting indicia in operative registry with the one eye of the patient upon which the surgical procedure is desired to be performed, and
unfolding the sheet of the incise drape about its fold line, while maintaining the sighting indicia in operative registry with the one eye of the patient so that the sheet substantially returns to undeformed, planar condition across the patient's face and so that by subsequently moving the opposing edges of the primary slit portion apart to form an opening in the sheet through which the desired surgical procedure is to be performed, the formed opening is substantially aligned with the one eye of the patient.

21. The method as defined in claim 20 wherein the step of holding is effected with one hand of the user and so that when said one eye of the patient is the right eye of the patient, the one hand is the right hand of the user, and so that when said one eye of the patient is the left eye of the patient, the one hand is the left hand of the user.

22. The method as defined in claim 21 wherein the step of placing includes the step of manipulating, with said one hand of the user, the lower eyelid of said one eye to a retracted condition with the incise drape, and is accompanied by a step of manipulating, with the other hand of the user, the upper eyelid of said one eye to a retracted condition.
